(19) United States
(12) Patent Application Publication
(10) Pub. No.: US 2026/0193439 A1
(43) Pub. Date: Jul. 9, 2026

(54) METHOD FOR EXTRACTION AND TRANSFORMATION BY HYDROLYSIS OF PHTALATES CONTAINED IN PVC PLASTICS

(71) Applicant: IFP Energies nouvelles, Rueil-Malmaison (FR)

(72) Inventors: Alexandra CHAUMONNOT, Rueil-Malmaison Cedex (FR); Jerome MAJCHER, Rueil-Malmaison Cedex (FR); Adrien MEKKI-BERRADA, Rueil-Malmaison Cedex (FR); David PASQUIER, Rueil-Malmaison Cedex (FR)

(73) Assignee: IFP Energies nouvelles, Rueil-Malmaison (FR)

(21) Appl. No.: 19/130,538

(22) PCT Filed: Nov. 9, 2023
(86) PCT No.: PCT/EP2023/081315

§ 371 Date: May 15, 2025

(30) Foreign Application Priority Data
Nov. 18, 2022 (FR) 2211987

(51) Int. Cl. C08J 11/08; B29B 17/04; C07C 51/09; C07C 51/43; C07C 67/48

(52) U.S. Cl. CPC C08J 11/08; B29B 17/0412; C07C 51/09; C07C 51/43; C07C 67/48; C08J 2327/06

(57) ABSTRACT

The present invention relates to a process for obtaining phthalic acid (PA) and a reusable target PVC plastic, from a PVC feedstock containing at least one phthalate, including:
a) a solid-liquid extraction of said PVC feedstock in contact with an organic extraction solvent, producing a liquid phase comprising said phthalate and a first solid phase comprising PVC plastic depleted in said phthalate;
b) the conversion of said phthalate from a) to PA by hydrolysis;
c) a solid-liquid extraction between said first solid phase and the liquid phase from a) and/or b) producing a solid stream of PVC plastic depleted in said phthalate;
d) a liquid-solid phase change of the PA producing a mixed stream of at least a second solid phase enriched in PA,
e) a solid-liquid separation between the solid PA and the aqueous phase of the mixed stream producing a solid stream of PA and an aqueous liquid effluent.

### TECHNICAL FIELD

The invention relates to the field of the recycling of plastics based on poly(vinyl chloride) (PVC), in particular to a process for extracting and converting phthalates, which are plasticizers included in the composition of PVC, by hydrolysis. More precisely, the invention relates to a process for recovering a phthalic acid (PA) and a reusable target PVC plastic from a PVC feedstock containing at least one phthalate.

### PRIOR ART

By definition, a plastic is a mixture consisting of a base polymeric material and numerous additives, the assembly being able to be moulded or fashioned (generally at elevated temperature and/or under pressure), so as to obtain a semifinished product or an object. A commonly accepted practice is to name said plastic by the name of the polymer of which it is made. Thus, the poly(vinyl chloride) (PVC) plastic in fact corresponds to the combination of the PVC polymer, referred to in the rest of the description as “PVC resin”, with various additives chosen on the basis of the functionalities required for said plastic. Said additives may be organic molecules or macromolecules or alternatively inorganic (nano) particles and are used as a function of the properties that they afford to the PVC resin: heat resistance, light resistance or resistance to a mechanical stress (stabilizers), flexibility (plasticizers), processability (lubricants), colouring (dyes/pigments), etc.

Several methods for recycling PVC plastics exist: “conventional” methods by simple mechanical recycling of the plastics, methods involving modifications of their composition, or even chemical conversion of the compounds of which they are made.

Since the middle of the 20th century, the recycling of PVC plastic involving a chemical action has been the subject of numerous studies directed, in a first step, toward dissolving the PVC resin with a variable proportion of additives and then, in a second step, toward recovering said resin using various chemical processes (precipitation, evaporation, etc.) in the presence of all or some of the soluble additives. For example, patents EP0945481, EP1268628 and EP2276801 are directed, respectively, toward recycling various PVC-based objects (flexible or rigid pipes, window frames, cables, etc.) and specifically fibre-reinforced PVC-based objects (tarpaulins, floor coverings, etc.) according to a process involving a first step of dissolving the PVC resin and the soluble additives in an organic solvent, followed by a second step of steam precipitation enabling the recovery of the resin and of the majority of the additives.

However, it is not always desirable to keep said additives in the PVC thus recovered to be recycled. For example, the changes over time in the regulations concerning them is a determining factor. Thus, certain plasticizers belonging to the phthalate family, which were notably widely used for formulating “flexible” PVCs about 40 years ago, gradually became subject to authorization in Europe on the basis of the REACH regulation, which, since the end of 2006, is directed toward establishing the safety of the manufacture and use of chemical substances in the European industry and, finally, were gradually excluded from the additives permitted for use. This is notably the case for the following nonexhaustive list of phthalates: dibutyl phthalate (DBP), dioctyl phthalate or diethyl hexyl phthalate (DOP or DEHP), benzyl butyl phthalate (BBP), diisobutyl phthalate (DIBP), dipentyl phthalate (DPP), diisopentyl phthalate, n-pentyl isopentyl phthalate, dihexyl phthalate, etc.

These new regulations are now leading toward the banning of the presence of such compounds in recycled raw materials (RRM). Taking into account the often very long service life of PVC-based objects (several decades), PVC-based objects formulated prior to the end of 2006 and now at the end of their service life cannot be recycled via regeneration methods that result in these banned additives being retained, whether said methods are conventional, such as mechanical recycling processes, or not, for instance the dissolution/precipitation process examples mentioned above.

Moreover, the phthalate plasticizers currently used in Europe (REACH-compatible phthalates) and in the rest of the world represent high value-added additives which are not upgraded when they are kept in the PVC recycled raw material. The reason for this is that they are expensive products, present in appreciable proportions in the initial PVC formulations (several tens of percent), and cannot directly give the PVC RRM the ad hoc flexibility properties. Supplying “fresh” plasticizers in appreciable amount is then essential for the reusability of the recycled PVC material.

The extraction of additives of phthalate type from PVC-based objects for removal or upgrading is thus a major challenge for optimized recyclability of PVC.

Several processes involving a step of dissolving the PVC resin have been adapted to enable this extraction. For example, patents EP1311599 and JP2007191586 both propose a first step of dissolving the PVC resin and at least phthalate-type additives with a first organic solvent, followed by a second step of liquid-liquid extraction of the phthalates from the solution obtained previously via the use of a second organic solvent different from the first organic solvent. Patent JP2007092035 discloses another possible example of implementation with dissolution of the PVC resin and at least phthalate-type additives via the use of a solvent under supercritical conditions and recovery of said phthalates in this same solvent after “rupture” of said supercritical conditions.

The removal or upgrading of phthalate-type additives from a PVC plastic may also be performed without proceeding via a preliminary step of dissolving said plastic, notably via direct extraction of said phthalates from the solid polymer matrix with a suitable organic solvent, as is fully indexed in the publication from Ugdüler et al., 2020, “Challenge and opportunities of solvent-based additive extraction methods for plastic recycling”, Waste Management, 104, 148-182. The challenge then lies in optimizing the extraction conditions (nature of the solvent, contact time, temperature, pressure, etc.) to achieve the best possible yields of extracted phthalates. Although this methodology for the removal of phthalates from PVC plastics is frequently used, notably for detecting and analytically quantifying these specific additives in said plastics, to the Applicant's knowledge, no process for regenerating PVC-based objects involves this technique.

Although critical for ensuring efficient recycling of PVC plastics and for obtaining a reusable recycled PVC, the extraction of phthalate-type plasticizers is insufficient to ensure the economic viability of a process for regenerating PVC-based objects. The main reason frequently put forward is the difficulty in finding an economically viable balance between the cost of the individual operations performed in said regeneration process and the resale cost (equivalent to the added value) of the products obtained. Said products consist of the phthalate-free PVC-based recycled material, which is naturally upgradable, and of said extracted phthalates which are, themselves, sparingly upgradable. Specifically, any regeneration process involving a step of extracting the phthalates from PVC-based objects will lead to the recovery of a mixture of phthalates which may comprise phthalates that are not “REACH-compatible”. The upgrading of said non-REACH-compatible phthalates is, of course, excluded and said phthalates will need to be treated as specific waste giving rise to additional costs. The upgrading of the REACH-compatible phthalates, which is advantageous per se, is in point of fact difficult since it involves technically complex and expensive separation/purification steps.

In the past, some studies focused on bringing phthalate-containing PVC plastics into contact with highly concentrated basic aqueous solutions (essentially NaOH) to convert said phthalates and extract the resulting product(s): a salt of phthalic acid and of possible degradation products depending on the associated operating conditions. This chemical reaction was carried out together with or upstream of a PVC dechlorination step, then making it possible to obtain a non-chlorinated residue that is predominantly free of phtalates to allow the energy recovery thereof. Carrying out such a step upstream of the dechlorination, and assisted by high frequencies or microwaves, has the advantage of recovering an upgradable phthalic acid salt, as has been reported in the following documents: patent JP3929352; the publication by F. Osada et al., 2010, “Deplasticization and dechlorination of flexible polyvinyl chloride in NaOH solution by microwave heating”, J. Mater. Cycles Waste Manag., 2010, 12, 245; the publication by S. M. Shin et al., 2011, “Elution Behavior of Additive Agent from Flexible PVC”, Chawon Rissaikuring, 10, 6, 3. However, this implementation has the following major drawbacks: it requires the use of highly concentrated bases, it results in the production not of phthalic acid but of the associated salt thereof, and the extraction of the phtalates is not optimized and does not meet the REACH regulation applicable since 2006 for the recovery of an upgradable compound as recycled raw material.

### SUMMARY OF THE INVENTION

The present invention is aimed at overcoming, at least partly, the problems of the prior art and is particularly directed toward providing a process for regenerating PVC-based objects allowing the treatment of any type of PVC feedstock containing phthalates and the conversion thereof into two products of interest that can be upgraded as raw materials: phthalic acid and a recyclable PVC plastic free of phthalates, notably of undesirable phthalates, typically those that are subject to authorization by the European REACH regulation.

The phthalic acid is notably used to manufacture phtalates, which are derivatives of phthalic acid. The phthalic acid can be used as raw material for the manufacture of other chemicals, in fields different from that of formulating plastics, for example for manufacturing dyes, fragrances, sweeteners such as saccharine, etc.

The process according to the invention makes it possible in particular to produce a phthalic acid powder of good purity from a PVC feedstock, typically a PVC waste, without stoichiometric consumption of base or acid.

Thus, to achieve at least one of the abovementioned objectives, among others, the present invention proposes, according to a first aspect, a process for recovering phthalic acid and a reusable target PVC plastic from a PVC feedstock containing at least one phthalate, including the following steps:
a) a solid-liquid extraction of said PVC feedstock in the form of particles by placing particles of the PVC feedstock in contact with at least one organic solvent for extracting said phthalate, to produce a liquid phase enriched in said phthalate and a first solid phase comprising PVC plastic depleted in said phthalate;
b) a chemical conversion of said phthalate extracted in step a) to phthalic acid of formula C₆H₄(COOH)₂ by hydrolysis using water to produce an aqueous phase comprising said phthalic acid;
c) a solid-liquid extraction between said first solid phase and said liquid phase enriched in said phthalate or said aqueous phase comprising said phthalic acid to produce at least one solid stream comprising the PVC plastic depleted in said phthalate in order to recover the target PVC plastic;
d) a phase change of the phthalic acid from the dissolved state in said aqueous phase to a solid state to produce a mixed stream comprising an aqueous liquid phase depleted in phthalic acid and a second solid phase enriched in phthalic acid;
e) a solid-liquid separation between the phthalic acid in the solid state from step d) and the aqueous liquid phase of said mixed stream, to produce a solid stream of phthalic acid and a liquid effluent comprising residual water from step b).

One advantage of the present invention lies in the ability of the process, by means of a solid-liquid extraction step that is compatible and combined with a chemical hydrolysis reaction, to extract and convert a mixture of phthalates initially trapped in polymeric matrices of various objects based on PVC plastic, irrespective of the composition of said mixture (i.e. irrespective of the nature and origin of the various phthalates) and despite the possible presence of numerous other additives, into a single REACH-compatible and upgradable phthalate product: phthalic acid. Obtaining the single phthalic acid product from the mixture of phtalates makes it possible, in addition, to consider the conversion of said phthalic acid into novel and multiple phtalates, still very widely used in many fields such as that of plastics processing, according to a circular economy principle.

According to a first variant, steps a) and b) are carried out within the same individual operation, producing a stream comprising at least the aqueous phase comprising phthalic acid and the first solid phase comprising PVC plastic depleted in said phthalate.

According to a second variant, alternative to the first variant, steps a) and b) form the subject of two distinct individual operations, step a) producing a stream comprising the liquid phase enriched in said phthalate and said first solid phase sent to step c) carried out between steps a) and b), step c) producing the stream comprising the PVC plastic depleted in said phthalate and a first liquid stream comprising said liquid phase enriched in said phthalate.

According to this second variant, the process preferably comprises a step g) for removing at least one portion of the organic extraction solvent from said first liquid stream prior to step b), to produce a first concentrated liquid stream comprising said phthalate sent to step b).

According to one or more embodiments, step g) of removing at least one portion of the organic extraction solvent from the first liquid stream comprises the evaporation of the extraction solvent, preferably using a series of flash drums.

According to one or more embodiments, the organic extraction solvent recovered in step g) is recycled at least in part to step a).

According to one or more embodiments, the solid stream comprising the PVC plastic depleted in phthalates separated in step c) is recycled at least in part to step a).

According to one or more embodiments, the organic extraction solvent is chosen from the list consisting of ketones, ethers, glycol ethers, cyclic and aromatic hydrocarbons, alcohols with a linear or branched short aliphatic chain, of empirical formula CₙH₂ₙ₊₁OH with n<4, n being a non-zero natural integer, and mixtures thereof.

According to one or more embodiments, the organic extraction solvent is chosen from the list consisting of methyl ethyl ketone, diethyl ketone, 4-heptanone, 2,4-dimethyl-3-pentanone, methyl isobutyl ketone, cyclopentanone, cyclohexanone, acetophenone, tetrahydrofuran, cyclohexane, xylenes, toluene, methanol, ethanol, n-propanol, i-propanol, and mixtures thereof, and preferably is chosen from said alcohols, ketones/alcohols mixtures, hydrocarbons/alcohols mixtures, cyclohexane, toluene, xylenes, methoxycyclopentane, methyl isobutyl ketone, cyclopentanone, acetophenone, and preferably said organic extraction solvent is toluene, methyl ethyl ketone, acetophenone or a methyl ethyl ketone/methanol mixture.

According to one or more embodiments, the hydrolysis in step b) is carried out in the presence of an acid hydrolysis catalyst, preferably a homogeneous acid catalyst chosen from the list consisting of mineral Brönsted acid catalysts, preferably hydrochloric acid, sulfuric acid or phosphoric acid, organic Brönsted acid catalysts, preferably p-toluenesulfonic acid, and Lewis acid catalysts, preferably AlF₃, or a heterogeneous acid catalyst chosen from the list consisting of aluminas, chlorinated aluminas, fluorinated aluminas, mesoporous aluminosilicates, zeolites and mixtures thereof with other oxides, (H+) ion-exchange resins, preferably sulfonic resins.

According to one or more embodiments, the hydrolysis in step b) is carried out at a temperature of between room temperature and 150° C., preferably between 40° C. and 130° C., at a pressure of between atmospheric pressure and 5.0 MPa, preferably between atmospheric pressure and 2.0 MPa, and for a time of between 1 minute and 10 hours, preferably between 10 minutes and 4 hours.

According to one or more embodiments, the hydrolysis in step b) is carried out so that the mole ratio of the amount of water to the amount of said phthalate extracted in step a) is between 100 and 9000.

According to one or more embodiments, step d) comprises at least one step of precipitating the phthalic acid, preferably comprising a cooling to a temperature between 10° C. and room temperature. According to one or more embodiments, said at least one phthalate of said PVC feedstock is a phthalate of empirical formula C₆H₄(COOR₁) (COOR₂) in which the ester groups are in the ortho position of the benzene ring, R₁ or R₂ being chosen independently from one of the elements of the group consisting of a linear or branched or cyclic alkyl chain, a linear or branched alkoxyalkyl chain, or an aryl or alkylaryl chain, R₁ and/or R₂ preferably comprising between 1 and 20 carbon atoms, or even between 1 and 15 carbon atoms.

According to one or more embodiments, the target PVC plastic is free of said phthalate, and preferably comprises less than 0.1% by mass in total of phthalates chosen from the list consisting of dibutyl phthalate, dioctyl phthalate or diethylhexyl phthalate, benzyl butyl phthalate, dibutyl phthalate, diisobutyl phthalate, dipentyl phthalate, diisopentyl phthalate, n-pentyl isopentyl phthalate, dihexyl phthalate, bis(2-methoxyethyl) phthalate, and mixtures thereof.

According to one or more embodiments, the process comprises an additional separation step f) directly upstream of step d) or downstream of step e) to separate at least one aqueous liquid phase from other compounds in the liquid or gas phase. When some of said compounds comprise extraction solvent or phthalate extracted in step a) and not converted and/or partially converted in step b) or water, they are preferably recycled: the extraction solvent can be recycled to step a), and the water and/or the phthalate extracted in step a) and not converted and/or partially converted in step b) can be recycled to step b).

According to a second aspect, the present invention relates to a process for recycling a PVC-based object containing at least one phthalate, including:
the conditioning of said PVC-based object comprising at least milling or shredding of said PVC-based object to form a PVC feedstock in the form of particles;
the recovery of phthalic acid and of a reusable target PVC plastic from said PVC feedstock in the form of particles according to the first aspect of the invention.

The present invention also relates, according to a third aspect, to a process for manufacturing a flexible PVC-based object comprising a recycled PVC plastic and/or a phthalate manufactured from phthalic acid recovered by the process according to the first aspect of the invention.

Other subjects and advantages of the invention will become apparent on reading the description which follows of particular implementation examples of the invention, which are given as nonlimiting examples, the description being made with reference to the appended figures described below.

### LIST OF FIGURES

FIG. 1 is a diagram of the process according to one embodiment of the invention comprising the steps a), b), c), d) and e), the optional step g) of removing the extraction solvent, and the optional separation step f1), and in which the extraction step a) and hydrolysis step b) form the subject of two distinct individual operations with the solid-liquid separation step c) carried out between steps a) and b) (second variant of the process according to the invention). The organic extraction solvent (7) is water-miscible.

FIG. 2 is a diagram of the process according to another embodiment comprising, like the embodiment illustrated in FIG. 1, separate steps of extraction a) and hydrolysis b), with an intermediate solid-liquid separation step c), and further comprising the optional step g), and optional separation steps f3) and f2) directly upstream of step d) and downstream of step e) respectively. The organic extraction solvent (7) is water-miscible.

FIG. 3 is a diagram of the process according to another preferred embodiment comprising, like the embodiments illustrated in FIGS. 1 and 2, separate steps of extraction a) and hydrolysis b), with an intermediate solid-liquid separation step c), and further comprising the optional step g), and an optional separation step f4) directly upstream of step d) and also an optional separation step f5) downstream of step f4). The organic extraction solvent (7) is water-immiscible.

FIG. 4 is a diagram of the process according to another embodiment of the invention comprising the steps a), b), c), d) and e), and the optional separation step f1), and in which the extraction step a) and hydrolysis step b) are carried out within the same individual operation (first variant of the process according to the invention). The organic extraction solvent (7) is water-miscible.

FIG. 5 is a diagram of the process according to another embodiment comprising, like the embodiment illustrated in FIG. 4, simultaneous steps of extraction a) and hydrolysis b), with a solid-liquid separation step c) directly downstream, and further comprising the optional separation steps f3) and f2) directly upstream of step d) and downstream of step e) respectively. The organic extraction solvent (7) is water-miscible.

FIG. 6 is a diagram of the process according to another embodiment comprising, like the embodiments illustrated in FIGS. 4 and 5, simultaneous steps of extraction a) and hydrolysis b), with a solid-liquid separation step c) directly downstream, and further comprising the optional separation step f4) directly upstream of step d) and also an optional separation step f5) downstream of step f4). The organic extraction solvent (7) is water-immiscible.

In the figures, the same references denote identical or equivalent elements.

### DESCRIPTION OF THE EMBODIMENTS

### Terminology

Certain definitions are given below, although further details regarding the objects defined hereinbelow may be given later in the description.

The term “PVC-based object” means an object, generally a consumer object, which comprises, and preferably consists of, at least one PVC plastic.

The term “poly(vinyl chloride) plastic”, also known as PVC plastic or simply PVC, means the combination of a PVC polymer, also known as PVC resin, with various additives chosen on the basis of of the functionalities required for the PVC plastic, which are themselves chosen on the basis of the intended applications.

Said PVC polymer is derived from the radical polymerization of vinyl chloride (VCM), which monomer is itself obtained from chlorine and ethylene. Depending on the implementation of said polymerization, four families of PVC resins may be used: 1) suspension PVC or S-PVC resins (suspension polymerization of VCM), 2) emulsion PVC or PVC “paste” resins (emulsion polymerization), 3) mass PVC or M-PVC resins (mass polymerization) and 4) superchlorinated PVC or C-PVC resins, obtained by superchlorination as a post-treatment on the preceding resins.

Said additives included in the composition of a PVC plastic may be organic molecules or macromolecules or alternatively inorganic (nano) particles and are used as a function of the properties that they afford to the PVC resin: heat resistance, light resistance or resistance to a mechanical stress (stabilizers), flexibility (plasticizers), processability (lubricants), coloring (dyes/pigments), etc.

The term “phthalates” means the group of chemicals formed by dicarboxylic esters of phthalic acid. They are composed of a benzene ring and of two carboxylic ester groups placed in the ortho position on the benzene ring. They may be described by means of the following formula:

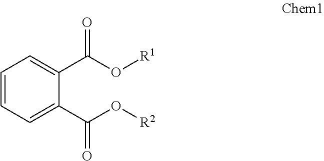

or alternatively by the empirical formula C₆H₄(COOR₁) (COOR₂), in which R₁ and R₂ are independently chosen from one of the elements of the group consisting of a linear, branched or cyclic alkyl chain, a linear or branched alkoxyalkyl chain, or an aryl or alkylaryl chain, it being possible for said alkyl, alkoxyalkyl, aryl or alkylaryl chain to typically comprise between 1 and 20 carbon atoms, or even comprise between 1 and 15 carbon atoms. For example, R₁ and/or R₂ may be chosen from ethyl, n-butyl, isobutyl, n-pentyl, isopentyl, n-hexyl, n-octyl, n-nonyl, isononyl, n-decyl, isodecyl, methoxyethyl and benzyl groups.

Phthalates are commonly used as plasticizers for plastics and in particular as plasticizers for plastics of PVC type, notably to make them flexible.

In the present description, the term “phthalic acid” (PA), also known under the name benzene-1,2-dicarboxylic acid or o-phthalic acid, denotes the product of empirical formula C₆H₄(COOH)₂ resulting from the hydrolysis reaction of at least one phthalate-type plasticizer (and in particular of empirical formula C₆H₄(COOR₁) (COOR₂), as described above) present in PVC-based objects with water (H₂O). The term “by-product(s) of alcohol type” (AL) means the by-product(s) of formula R₁OH or R₂OH resulting from the hydrolysis reaction of at least one phthalate-type plasticizer present in PVC-based objects with H₂O. R₁ and R₂ are defined identically to R₁ and R₂ of the phthalates.

The term “intermediate alkyl phthalate” (IAP) or “partially converted phthalate” means the by-product of empirical formula C₆H₄(COOH) (COOR₁) or C₆H₄(COOR₂) (COOH) resulting from the incomplete hydrolysis reaction of at least one phthalate-type plasticizer (and in particular of empirical formula C₆H₄(COOR₁) (COOR₂) as described above) present in PVC-based objects with H₂O. R₁ and R₂ are defined identically to R₁ and R₂ of the phthalates.

The term “reusable target PVC plastic” means a “phthalate-free PVC”, i.e. the solid comprising at least the PVC resin supplemented with at least one of the additives initially present in the PVC plastic of the PVC feedstock treated according to the invention, and from which the phthalates have been extracted and converted according to the invention to phthalic acid completely or partly. The term “phthalate-free” in particular means that the solid PVC obtained as product of the process according to the invention contains, in total, less than 0.1% by weight of phthalates subject to authorization by the REACH regulation in Europe (Annex XIV of Regulation (EC) No 1907/2006 of the European Parliament and of the Council of 18 Dec. 2006), in particular less than 0.1% by weight of phthalates chosen from the list consisting of the following phthalates: dibutyl phthalate (DBP), dioctyl phthalate or diethylhexyl phthalate (DOP or DEHP), benzyl butyl phthalate (BBP), dibutyl phthalate (DBP), diisobutyl phthalate (DIBP), dipentyl phthalate (DPP), diisopentyl phthalate, n-pentyl isopentyl phthalate, dihexyl phthalate, bis(2-methoxyethyl) phthalate, alone or as a mixture.

In the present description, the term “greater than . . . ” is understood as strictly greater than, and is symbolized by the sign “>”, and the term “less than . . . ” is understood as strictly less than, and is symbolized by the sign “<”.

In the present description, the term “room temperature” (r.t.) means a temperature typically of 20° C. ±5° C., and the term “atmospheric pressure” means a pressure of 0.101325 MPa.

In the present description, the term “comprise” is synonymous with (means the same as) “include” and “contain”, and is inclusive or open-ended and does not exclude other elements which are not specified. It is understood that the term “to comprise” includes the exclusive and closed term “to consist”.

In the present description, the expression “of between . . . and . . . ” means that the limiting values of the interval are included in the described range of values, unless otherwise specified.

In the present description, the various ranges of parameters for a given step, such as the pressure ranges and the temperature ranges, may be used alone or in combination. For example, in the present description, a range of preferred pressure values can be combined with a range of more preferred temperature values.

In the continuation of the text, specific embodiments of the invention may be described. They can be implemented separately or combined together, without limitation of combinations when this is technically feasible.

The description below of the process according to the first aspect of the invention refers to the diagrams in FIGS. 1 to 6, illustrating various implementations of the process according to the invention.

In accordance with the invention, the process for recovering PA and a reusable target PVC plastic from a PVC feedstock containing at least one phthalate includes, and may consist of, the following steps:
a) a solid-liquid extraction of said PVC feedstock in the form of particles 1 by placing particles of the PVC feedstock in contact with at least one organic solvent 7 for extracting said phthalate, to produce a liquid phase enriched in said phthalate and a first solid phase comprising PVC plastic depleted in said phthalate;
b) a chemical conversion of said phthalate extracted in step a) to phthalic acid of formula C₆H₄(COOH)₂ by hydrolysis using water 9 to produce an aqueous phase comprising said phthalic acid;
c) a solid-liquid extraction between said first solid phase and said liquid phase enriched in said phthalate or said aqueous phase comprising said phthalic acid to produce at least one solid stream comprising the PVC plastic depleted in said phthalate 8 in order to recover the target PVC plastic;
d) a phase change of the phthalic acid from the dissolved state in the aqueous phase to a solid state to produce a mixed stream comprising an aqueous liquid phase depleted in phthalic acid and a second solid phase enriched in phthalic acid (5, 19, 22);
e) a solid-liquid separation between the phthalic acid in the solid state from step d) and the aqueous liquid phase of said mixed stream, to produce at least a solid stream of phthalic acid 6 and a liquid effluent (11, 14, 20) comprising residual water from step b).

### Feedstock

The process according to the invention is fed with a feedstock known as “PVC feedstock” 1 comprising at least one PVC plastic, which necessarily comprises at least one phthalate as described in the present invention.

Said PVC plastic may include at least 0.1% by weight of phthalates, or even at least 1% by weight of phthalates or else at least 5% by weight of phthalates. In general, the PVC plastics advantageously comprise less than 60% by weight of phthalates, typically less than 40% by weight of phthalates.

Said PVC feedstock is advantageously a feedstock of PVC to be recycled of the “production scraps” type, i.e. waste resulting from the processes for producing the PVC polymer during its polymerization or the PVC plastic during its formulation/forming or the PVC-based object during its production, or of the “post-consumer waste” type, i.e. waste generated after use of said PVC-based object by the user.

In particular, the PVC feedstock to be recycled may be derived from any existing collection and sorting channels or networks for production scraps and/or post-consumer waste making it possible to isolate a stream based on at least one PVC plastic comprising at least one phthalate, notably the collection and sorting channels or networks specific to plastic waste.

Thus, the PVC feedstock, which is typically of “production scraps” type and/or of “post-consumer waste” type, generally comes from the main fields of application which use PVC plastic, such as, in a nonexhaustive manner, the following fields: building and construction, packaging, motor vehicles, electrical and electronic equipment, sports, medical equipment, etc. Preferably, the PVC feedstock comes from the building and construction field. More precisely, the PVC-based objects are generally used in these fields as various rigid profiles (windows, doors, blinds, roller shutter boxes), pipes and connections, and rigid bottles, plates and films, flexible films and sheets, flexible tubes and profiles, cables, floor coverings, coated fabrics, etc. Preferably, the PVC-based objects forming the PVC feedstock comprise at least “flexible” PVC, i.e. PVC containing additives of plasticizer type, preferably of phthalate type, as is the case, for example, for the following PVC-based objects: flexible films and sheets, flexible tubes and profiles, cables, floor coverings, coated fabrics, etc.

Advantageously, the PVC feedstock comprises at least 50% by mass, preferably at least 70% by mass, preferably at least 90% by mass and even more preferably at least 95% by mass of PVC plastic comprising at least one phthalate.

Preferably, the PVC feedstock comprises “flexible” PVC, i.e. PVC containing additives of plasticizer type, preferably of phthalate type.

Even more preferably, the PVC feedstock predominantly or even exclusively comprises “flexible” PVC, i.e. PVC containing additives of plasticizer type, preferably of phthalate type.

The PVC feedstock treated in the process for recovering a DAP and a reusable target PVC plastic according to the invention is in the form of particles. Thus, if the PVC feedstock is in an initial form which is that specific to production scraps or post-consumption waste, notably, in the latter case, in the initial form of PVC-based objects, it may first undergo a conditioning step comprising at least milling or shredding to form a PVC feedstock in the form of particles. Depending on the channels and/or networks from which these production scraps and/or PVC-based objects at the end of their service life are derived, the PVC waste may be milled and/or washed and/or may undergo any other conditioning step as described hereinbelow, so as to form the PVC feedstock in the form of particles that are suitable for the process according to the invention. For example, the PVC feedstock may advantageously be in the form of milled and optionally washed material, the largest dimension of which is less than 20 cm, preferably less than 10 cm, preferably less than 1 cm and even more preferably less than 5 mm. The PVC feedstock may also advantageously be in micronized solid form, i.e. in the form of particles preferably having a mean size of less than 1 mm, for example between 10 micrometers (μm) and 800 micrometers (μm). The mean size advantageously corresponds to the mean diameter of the spheres in which said particles are circumscribed.

Thus, the term “PVC feedstock in the form of particles” means particles of PVC plastic typically having a mean size, as defined previously, of between 10 μm and 20 cm, for example particles of milled material type with a mean size of between 1 mm and 20 cm, preferably between 1 mm and 10 cm, more preferentially between 1 mm and 1 cm, even more preferentially between 1 mm and 5 mm, or particles derived from micronization (very fine milling to produce a powder) with a mean size of less than 1 mm, preferably between 10 μm and 800 μm.

Preferably, the PVC feedstock treated in the process according to the invention is in the form of particles of milled material type, preferably particles with a mean size of between 1 mm and 5 mm, or particles derived from micronization (very fine milling to produce a powder) with a mean size of less than 1 mm.

The PVC feedstock may also comprise “macroscopic” impurities, such as glass, metal, plastics other than PVC (for example PET, etc.), wood, paper, cardboard, mineral elements, etc. Advantageously, the PVC feedstock comprises at most 50% by weight, preferably at most 30% by weight, preferably at most 10% by weight and even more preferably at most 5% by weight of “macroscopic” impurities.

The various steps of the process according to the invention leading to the PA and to the reusable target PVC plastic are detailed in the paragraphs that follow.

### Optional Preliminary Step of Conditioning the PVC Feedstock

According to the invention, the process may comprise a preliminary step of conditioning the PVC feedstock (not shown in the figures) including at least one step of milling or shredding the PVC feedstock to form a PVC feedstock in the form of solid particles as defined above, which can be sent into the solid-liquid extraction step a). This preconditioning step may also comprise one or more steps mentioned in the following non-exhaustive list: milling by micronization, sorting, oversorting, washing, drying, etc. Depending on the nature of the PVC feedstock treated, the step or steps, and their possible frequencies and sequences, involved in the preconditioning step are notably chosen by a person skilled in the art so as to limit the amount of macroscopic impurities and to reduce the size of the solid elements of which the PVC feedstock is initially composed.

For example, the preconditioning step makes it possible to provide a PVC feedstock in the form of particles, for example washed milled material with a mean size of less than 5 mm, the macroscopic impurity content of which is preferably at most 10% by weight and more preferably at least 5% by weight. Said preconditioned PVC feedstock may also be in the form of micronized solid particles, i.e. in the form of particles with a mean size of less than 1 mm, for example between 10 μm and 800 μm.

### Step a) of Solid-Liquid Extraction of the Phthalates

The process according to the invention comprises a step a) of solid-liquid extraction of the phthalate(s) from the PVC feedstock in the form of particles 1 by placing said feedstock in contact with an organic extraction solvent 7, so as to obtain an effluent comprising at least a liquid phase and a first solid phase. Said liquid phase is then enriched in said phthalate(s), and the first solid phase comprises PVC plastic depleted in said phthalate(s). The effluent is represented by the stream 2 in FIGS. 1 to 3, where steps a) and b) are performed separately.

Said organic extraction solvent 7 is thus chosen owing to its physicochemical properties for its ability to penetrate into the polymer matrix while drastically limiting the dissolution thereof, in order to effectively extract the phthalate(s) from the PVC feedstock in the form of particles. For this, a person skilled in the art may rely on knowledge of the Hildebrand and/or Hansen solubility parameters of the solvents to define, relative to these same parameters specific to the PVC resins and to the phtalates, the most appropriate solvent for carrying out the solid-liquid extraction step a) of the process according to the invention.

Hansen's theory makes it possible for example to estimate the solubility of a polymer, in particular of a thermoplastic such as PVC, in a solvent or a mixture of solvents, owing to the determination of the Hansen solubility parameters and sphere respectively for the solvent and the polymer. These calculations are based on the estimation of the cohesive forces enabling a compound (here the polymer) to remain in the solid state and which are split into 3 contributions: London interactions, Keesom interactions and hydrogen bonds. If a solvent or mixture of solvents has Hansen parameters in the Hansen sphere of the PVC polymer, said PVC polymer should be at least partially, preferably entirely, soluble in said solvent. Thus, a person skilled in the art will know how to use these calculations to choose an appropriate extraction solvent (i.e. organic solvents/mixture of organic solvents), which can solubilise the phthalates, while limiting the dissolution of the PVC polymer, which can then be the subject of an experimental verification (for a set of given operating conditions).

Said extraction solvent is also chosen so as to enable the implementation of step b) of chemical conversion of said phthalate(s) by hydrolysis, while limiting the secondary chemical reactions and while making the subsequent separation step(s), necessary for obtaining the PA according to the invention, simpler and more effective.

According to one or more embodiments, said extraction solvent is advantageously an organic solvent or mixture of organic solvents, chosen from:
ketones, such as methyl ethyl ketone (MEK), diethyl ketone (DEK), 4-heptanone, 2,4-dimethyl-3-pentanone, methyl isobutyl ketone (MIBK), cyclic ketones such as cyclopentanone and cyclohexanone, and aromatic ketones such as acetophenone,
ethers such as methoxycyclopentane (CPME), cyclic ethers such as tetrahydrofuran (THF), glycol ethers such as 2-methoxyethanol, ethylene glycol monobutyl ether, diethylene glycol monomethyl ether, diethylene glycol dimethyl ether and diethylene glycol monoethyl ether,
cyclic and aromatic hydrocarbons such as cyclohexane, xylenes and toluene,
alcohols with a linear or branched short aliphatic chain, of empirical formula CₙH₂ₙ₊₁OH with n<4, n being a non-zero natural integer, such as methanol, ethanol, n-propanol and i-propanol.

Preferably, the organic extraction solvent is chosen from the abovementioned alcohols, in particular methanol, ethanol, n-propanol, i-propanol, the abovementioned ketone/alcohol mixtures, the abovementioned hydrocarbon/alcohol mixtures, cyclohexane, toluene, xylenes, CPME, MEK, MIBK and cyclopentanone, taken alone or as a mixture, and more preferentially chosen from the abovementioned alcohols, the abovementioned ketone/alcohol mixtures, the abovementioned hydrocarbon/alcohol mixtures, cyclohexane, toluene, xylenes, acetophenone, MEK, MIBK and cyclopentanone, taken alone or as a mixture.

The extraction solvent may advantageously be toluene, MEK, acetophenone, or an MEK/methanol mixture.

Step a) of solid-liquid extraction of the phthalate(s) from the PVC feedstock 1 is preferably performed under the following operating conditions: a temperature of between room temperature and 200° C., preferably between 40° C. and 180° C., more preferably between 60° C. and 150° C., and even more preferentially between 60° C. and 145° C., a pressure of between atmospheric pressure and 11.0 MPa, preferably between atmospheric pressure and 5.0 MPa, more preferably between atmospheric pressure and 2.0 MPa, a residence time of between 1 minute and 10 hours, preferably between 5 minutes and 4 hours, more preferably between 5 minutes and 2 hours and even more preferably between 10 minutes and 30 minutes.

Preferably, step a) is performed so that the mole ratio of the amount of solvent 7 to the amount of the phthalate(s) to be extracted from the PVC feedstock 1 is between 2 and 250, preferably between 4 and 100 and even more preferably between 4 and 30.

The reactor used in step a) of the process according to the invention may advantageously be a stirred reactor which is stirred by a mechanical stirring system and/or by a recirculation loop and/or by fluidization, and/or with ultrasound, for example a batch or continuous reactor, which is preferably perfectly stirred, or a rotary drum reactor.

As regards the implementation, the PVC feedstock in the form of particles 1 and the organic extraction solvent 7 are advantageously mixed.

According to a first option, said mixing may be performed prior to the introduction of the PVC feedstock and the extraction solvent into the reactor of the solid-liquid extraction step a). In this case, said mixture may be formed in a mixer and may then be introduced into the reactor, said reactor being maintained at a desired pressure and temperature.

According to a second option, the PVC feedstock in the form of particles 1 and the solvent 7 may be introduced separately into the reactor of step a) of the process according to the invention. Said solid PVC feedstock and the solvent are then preferably injected into the reactor via two separate lines, one allowing the injection of the extraction solvent 7 and the other the solid PVC feedstock in the form of particles 1. In this case, the mixture of the PVC feedstock and of the solvent forms directly in said reactor.

In accordance with the invention, said solid-liquid extraction step a) makes it possible to obtain at least one effluent comprising at least a liquid phase containing at least the extracted phthalates and at least a first solid phase containing the PVC plastic depleted in phthalates, preferably free of phthalates.

Under the operating conditions of this step, the phthalate(s) extracted are advantageously in a liquid form.

### Step b) of Chemical Conversion of Said Phthalates by Hydrolysis

The process according to the invention comprises a step b) of chemical conversion of the phthalate(s) extracted in step a) to give at least the phthalic acid of formula C₆H₄(COOH)₂ by hydrolysis reaction, preferably in the liquid phase, between said phthalate(s) extracted in step a) and water (H₂O). Step b) of hydrolysis of the phthalate(s) present in order to form phthalic acid is preferably performed under the following operating conditions: a temperature of between room temperature and 150° C., preferably between room temperature and 145° C., more preferentially between 40° C. and 130° C., and more preferably between 60° C. and 110° C., a pressure of between atmospheric pressure and 5.0 MPa, preferably between atmospheric pressure and 2.0 MPa, more preferably between atmospheric pressure and 0.5 MPa, a residence time of between 1 minute and 10 hours, preferably between 10 minutes and 4 hours, more preferably between 10 minutes and 2 hours and even more preferably between 10 minutes and 1 hour.

Water 9 is therefore introduced into this step b) of the process, in order to carry out the hydrolysis reactions of the phthalate(s) so as to form phthalic acid.

Preferably, step b) is performed so that the mole ratio of the amount of water 9 to the amount of phthalates to be converted of the liquid phase containing the phthalate(s) extracted on conclusion of step a) is between 100 and 9000, preferably between 150 and 1800 and even more preferably between 200 and 850.

Preferably, said hydrolysis step b) is carried out in the presence of a hydrolysis catalyst 10, advantageously introduced into the reaction medium.

The hydrolysis catalyst 10 thus used is advantageously an acid catalyst, for example chosen from the acid catalysts of the following non-exhaustive list, which is well known to those skilled in the art, and preferably from the list consisting of:
homogeneous catalysts such as mineral Brönsted acid catalysts (e.g. hydrochloric acid, sulfuric acid, phosphoric acid, etc.), organic Brönsted acid catalysts (e.g. methanesulfonic acid, trifluoromethanesulfonic acid, trifluoroacetic acid, p-toluenesulfonic acid, etc.), and Lewis acid catalysts (e.g. AlF₃);
heterogeneous acid catalysts such as aluminas, chlorinated or fluorinated aluminas, mesoporous aluminosilicates, zeolites and mixtures thereof with other oxides, (H+) ion-exchange resins, for instance sulfonic resins, etc.

For example, the catalyst used according to the invention is a homogeneous catalyst, notably a homogeneous catalyst of organic Brönsted acid catalyst type such as p-toluenesulfonic acid.

Preferably, the amount of catalyst introduced is such that the weight ratio of the catalyst to the phthalate(s) to be converted is between 0.02% and 10% by weight, preferably between 1% and 8% by weight mass and even more preferably between 1% and 5% by weight.

Whether it is homogeneous or heterogeneous, the catalyst may be recycled and/or removed in the process according to methods that are well known to those skilled in the art, and is preferably recycled. It may be isolated, to be removed or preferably recycled for the hydrolysis reaction, in the downstream steps of the process or in any other dedicated step.

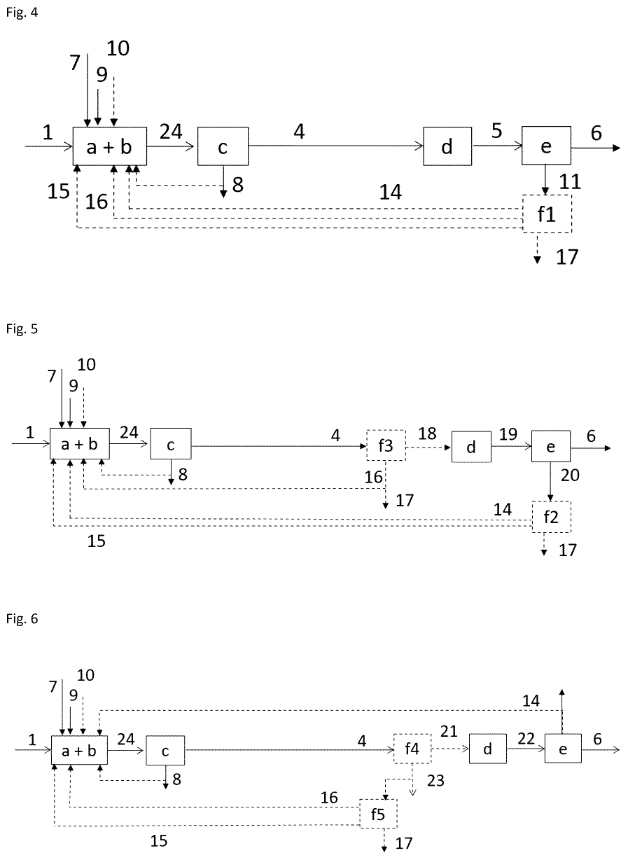

The reactor used in step b) may advantageously be a stirred reactor which is stirred by a mechanical stirring system and/or by a recirculation loop and/or by fluidization, and/or with ultrasound, for example a batch or continuous reactor, which is preferably perfectly stirred, or a rotary drum reactor. In accordance with the invention, said step b) of converting the phthalates makes it possible to obtain an effluent (stream 4 in FIGS. 1 to 3 or stream 24 in FIGS. 4 to 6) comprising at least one aqueous phase containing at least the phthalic acid obtained after hydrolysis reaction of the phthalates extracted in step a), which were initially contained in the liquid phase formed in step a).

Steps a) and b) of the process according to the invention may be carried out within the same individual operation upstream of the solid-liquid separation step c) of the process according to the invention, as represented in FIGS. 3 to 6, or else form the subject of two distinct individual operations that are separated by at least said step c), the individual operation of step a) then always being carried out before the individual operation of step b), as represented in FIGS. 1 to 3, and explained in greater detail below.

### Step c) of Solid-Liquid Separation

The process according to the invention comprises a step c) of solid-liquid separation between on the one hand the first solid phase containing the PVC plastic depleted in phthalates, preferably free of phthalates, and on the other hand the liquid phase containing the phthalate(s) extracted in step a) the aqueous phase comprising the phthalic acid obtained in step b), according to the first and second variants of the process described in detail below, and as a function of the position of step c) relative to steps a) and b).

The physical separation of the liquid and solid phases may advantageously be performed according to the techniques known to those skilled in the art, such as, in a non-exhaustive manner, filtration, centrifugation, for example the use of hydrocyclones, electrostatic precipitation or decantation, said techniques being used alone or in combination, in any order.

This step c) of solid-liquid separation thus makes it possible to produce at least one solid stream 8 comprising the PVC plastic depleted in the phthalate(s) extracted in step a), so as to recover said reusable target PVC plastic.

The production of the reusable target PVC as defined according to the invention may necessitate returning all or a portion of the solid stream 8 obtained in step c) into step a), in as many cycles as necessary so as to produce said target PVC plastic.

This possibility of recycling the solid stream is shown in FIGS. 1 to 6.

For example, step c) may take place by centrifugation of the liquid effluent 2 comprising the liquid phase from step a) containing at least the extracted phthalates and the first solid-phase, leading to the separation of said solid 8, and advantageously to the return of all or a portion of said solid into step a), preferably placed in suspension beforehand, for example by means of supplying extraction solvent 7 (not shown in the figures), until the reusable target PVC plastic is produced.

According to a first variant of the process according to the invention, the solid-liquid separation step c) takes place after performing step a) and before performing step b). This first variant is illustrated in FIGS. 1 to 3. In this case, the liquid effluent 2 from step a) is sent into the solid-liquid separation step c) which leads to separation of the liquid phase containing the extracted phthalates from the first solid phase containing the PVC depleted in phthalate(s). Step c) therefore produces the solid stream 8 comprising the PVC plastic depleted in phthalate(s), and a first liquid stream 3 which contains the phthalate(s) extracted in step a) and which is then sent to step b) for the conversion of said phthalate(s) by hydrolysis or to the optional step g) described below in the present invention enabling the complete or partial removal of the extraction solvent before sending the phtalates to be converted in step b). This first variant is particularly suitable in the case where the PVC feedstock to be treated would lead to the formation, during step a), of a solid phase that is unfavourable for performing the chemical hydrolysis reaction (in terms of chemical or rheological properties, etc.). A preferred example of implementation according to this variant is illustrated in FIG. 3.

According to a second variant of the process according to the invention, the solid-liquid separation step c) takes place after performing steps a) and b) carried out within the same individual operation. This second variant is illustrated in particular in FIGS. 4 to 6. In this case, the liquid effluent 24 from the simultaneous steps a) and b) is sent into the solid-liquid separation step c) which leads to separation between at least the aqueous phase containing at least the PA obtained after hydrolysis reaction in step b), and the first solid phase containing the PVC plastic depleted in phthalate(s). The (aqueous and organic) liquid phase and the first solid phase containing the PVC plastic depleted in phthalate(s) are separated on conclusion of this step c). The simultaneous (joint) implementation, within the same individual operation, of steps a) and b) leads to a reduction in the number of individual operations required for performing the process according to the invention and thus to a limitation of the number of items of equipment, of the amount of energy used, etc. and thus to a reduction of the costs. A preferred example according to this variant is illustrated in FIG. 5, the simultaneous implementation of steps a) and b) being depicted by the use of a single step (a+b) (a single “box” (a+b) represented). In this case, the presence of water 9 during the extraction phase may advantageously modify the extraction properties of the solvent 7 that it would have had when being used alone. For example, the combination of the extraction solvent 7 and water 9 may make it possible to use an extraction solvent 7 initially suitable for the extraction of the phtalates but having too high a solubilizing power for the PVC resin, and which is thus counterbalanced by the presence of water, the mixture ultimately having a lower solubilizing power for the PVC resin.

Furthermore, for the second variant, a temperature above or equal to room temperature and below or equal to 150° C., preferably below or equal to 145° C., and more preferentially below or equal to 100° C., is preferred in order to prevent any degradation of the PVC resin, for example by dechlorination reaction.

On conclusion of all of steps a), b) and c), the PA is mainly in liquid form in an aqueous phase contained in the liquid effluent 4. Said liquid effluent 4 may contain one or more liquid phases, for example a single liquid phase (single-phase liquid) or two liquid phases (two-phase liquid), in particular depending on the nature of the extraction solvent (example water-miscible or water-immiscible) and/or on the operating conditions chosen.

Step d) of liquid-solid-phase change of the phthalic acid

The process according to the invention comprises a step d) of phase change of the PA from the dissolved state in the aqueous phase obtained on conclusion of all of steps a), b) and c) (liquid effluent 4) to a solid state enabling it to be recovered in a subsequent solid-liquid separation step e). This liquid-solid-phase change may advantageously be performed by means of one or more crystallization or precipitation operations according to the techniques known to those skilled in the art, such as, in a non-exhaustive manner, cold wall crystallization, the use of a precipitating agent, a batch distillation, etc., said techniques being used alone or in combination, in any order.

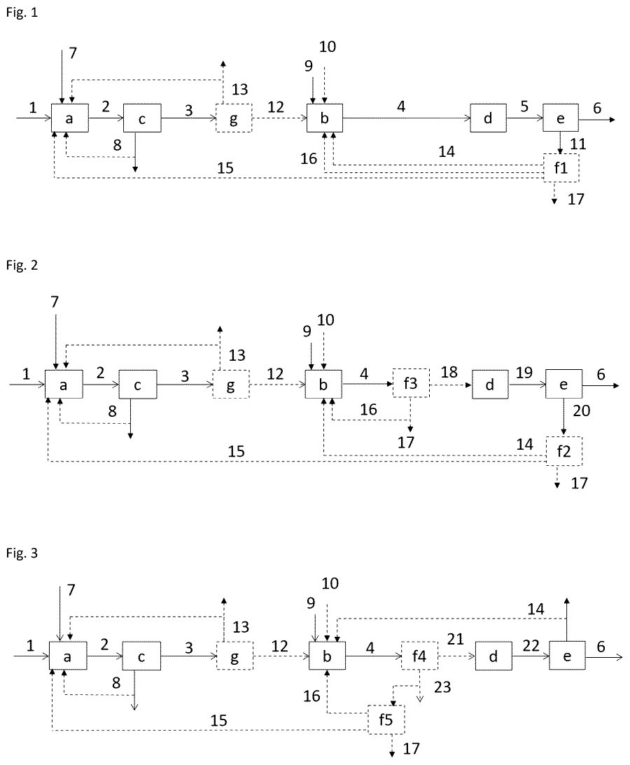

This step d) of liquid-solid-phase change therefore makes it possible to produce at least one mixed stream comprising an aqueous liquid phase depleted in phthalic acid and a second solid phase enriched in phthalic acid. This is stream 5 in FIGS. 1 and 4, stream 19 in FIGS. 2 and 5, and stream 22 in FIGS. 3 and 6.

In the embodiment represented in FIG. 3, which is one of the preferred embodiments according to the invention, the PA-enriched aqueous liquid effluent 21 is for example cooled to a temperature between 10° C. and room temperature, for example a temperature of 15° C., via the use of a cold-wall crystallization so as to give rise to the precipitation of the PA to obtain the mixed stream 22 comprising PA in the solid state.

Step e) of solid-liquid separation for the recovery of PA

The process according to the invention comprises a step e) of solid-liquid separation carried out on the mixed stream (5, 19 and 22 depending on the embodiments) from step d), comprising a second solid phase enriched in phthalic acid (phthalic acid in the solid state), to produce at least a solid stream comprising the PA 6 so as to recover the PA and a liquid effluent (11, 14 or 20 depending on the embodiments) comprising residual water from the hydrolysis step b).

The residual water may still comprise phthalic acid dissolved at low concentration.

Th PA of the solid stream 6 is in powder form.

The solid-liquid physical separation of the mix stream from step d) may advantageously be performed according to the techniques known to those skilled in the art, such as, in a non-exhaustive manner, filtration, centrifugation, for example the use of hydrocyclones, electrostatic precipitation or decantation, said techniques being used alone or in combination, in any order.

In the embodiment represented in FIG. 3, which is one of the preferred embodiments according to the invention, the mixed effluent 22 is for example treated by centrifugation so as to obtain the solid stream comprising the PA 6 and an aqueous stream 14, which is preferably completely or partly sent back to step b) of the process according to the invention.

According to one or more embodiments (not represented in the figures), steps d) and e) are carried out within the same individual operation.

It is possible to produce phthalic anhydride by sending the solid stream of PA 6 to a dehydration step, it being possible for the phthalic anhydride to be the starting compound taken for synthesising the phthalates of the PVC. The dehydration of PA to form phthalic anhydride is known, and such a dehydration step may be carried out as is for example described in patent U.S. Pat. No. 3,720,692. Step g) of removing the extraction solvent before the (optional) step c)

According to the first variant of the process of the invention, the process according to the invention preferably comprises a step g) enabling the removal of at least one portion of the extraction solvent 7, and at best all of said solvent, used during step a). This step g) is carried out downstream of the solid-liquid separation step c) and upstream of step b) of the process according to the invention, as illustrated for example in FIGS. 1 to 3). The removal, at most complete removal, of the extraction solvent enables a liquid effluent 12 to be obtained which is at a minimum concentrated in the phthalate(s) extracted in step a) and at best free of the extraction solvent. This liquid stream concentrated in said phtalate 12 is sent to step b). This step g), while making it possible to continue to bring about a liquid effluent having a rheology compatible with the various embodiments according to the invention, favours the hydrolysis reaction involved in step b) by a suitable shift of the thermodynamic equilibrium of said reaction in the direction of the formation of PA. Furthermore, the extraction solvent thus removed can be recovered and sent back at least to step a) of the process according to the invention.

Preferably, only a portion of the extraction solvent is removed in this step.

The removal of a portion or all of the extraction solvent 7 is advantageously carried out by evaporation according to techniques known to those skilled in the art such as distillation, evaporation, liquid-liquid separation, etc., said techniques being used alone or in combination, in any order. Step g) may thus comprise a gas-liquid separation, preferably the evaporation of the solvent, for example using a series of flash drums.

When the extraction solvent comprises at least one alcohol with a linear or branched short aliphatic chain, of empirical formula CₙH₂ₙ₊₁OH with n<4, said extraction solvent is preferably removed.

In the embodiment represented in FIG. 3, which is one of the preferred embodiments according to the invention, the liquid stream 3 containing the phthalate(s) extracted in step a) and resulting from step c) is treated by evaporation during the optional step g) so as to obtain the liquid stream 12 concentrated in said phthalate(s) which is itself sent to step b). The extraction solvent is typically a water-immiscible solvent, for example toluene, and is not completely removed during step g), which makes it possible, in the downstream steps of the process, to benefit from this immiscible nature for the separation of the components of the stream 12, and notably of the phthalic acid from the other compounds. The extraction solvent thus recovered is preferably recycled, at least in part, in the form of the stream 13, to step a).

### (Optional) Separation Step(s) f)

Depending on the choice of the extraction solvent and the various embodiments of the process according to the invention, notably with integration of the optional step g) described above, a liquid effluent 4 enriched in at least PA is obtained on conclusion of step b) of said process. This liquid effluent 4 comprises an aqueous phase comprising PA. The chemical nature and the amount of the other constituents of said stream vary as a function of the embodiments chosen and may have an impact on the nature of the medium, typically the liquid effluent 4 may be single-phase or two-phase. The process according to the invention may thus comprise one or more optional steps of liquid-liquid and/or liquid-gas separation located upstream and/or downstream of steps d) and e) of the process according to the invention in order to:
favour the implementation of steps d) and e), and therefore the production of at least one solid stream comprising PA 6 in order to recover PA, and/or
recovering, and sending back to the process, the residual water resulting from the hydrolysis step b) (stream 14) and the extraction solvent (stream 15), and/or
recovering, and sending back to the process, the phthalates possibly not converted and/or partially converted resulting from the hydrolysis step b), and/or
recovering alcohols, by-products of the chemical hydrolysis reaction, which may be upgraded or burnt to produce a portion of the energy needed for the process according to the invention.

The optional separation steps f) may be performed according to methods that are well known to those skilled in the art, such as, in a non-exhaustive manner, distillation, decantation, evaporation, liquid-liquid extraction, etc., performed alone or in combination. The operating conditions of this or these step(s) (temperature, pressure, etc.) are determined as a function of the chosen separation method.

In particular, one or more separation steps f) (cf. steps f1) to f4) described below) are carried out directly upstream of step d) or downstream of step e) to separate at least one aqueous liquid phase (i.e. which contains water) from other compounds in the liquid or gas phase.

Some of said compounds which comprise extraction solvent, or phthalate extracted in step a) and not converted and/or partially converted in step b), or water, may advantageously be recycled respectively to step a) or to step b).

In FIGS. 1 to 6, various separation steps f1), f2), f3), f4) and f5) are represented, and described in greater detail below.

Steps f1) and f2) are carried out directly downstream of step e). Steps f3) and f4) are carried out directly upstream of step d). Step f5) is a separation step directly downstream of the separation step f4).

According to one or more embodiments of the invention, and provided that the extraction solvent has not been completely removed from stream 3 via step g) when it is carried out, said solvent and the operating conditions of the process of the invention are chosen so that said solvent and the water 9 form one and the same liquid phase of the effluent 4 containing at least PA, the optional other components: phthalates extracted in step a) and not converted, IAP, and AL.

According to this or these embodiment(s), represented in FIGS. 1 and 4, the streams obtained on conclusion of step e) comprise the solid stream comprising PA 6 and a liquid effluent 11 comprising residual water (from step b)), said liquid effluent 11 being in the form of a single-phase liquid phase. The choice of a water-miscible solvent, such as MEK or a MEK/methanol mixture, may make it possible to obtain a single-phase liquid phase for effluents 4 and 11 resulting respectively from steps b) and e) of the process.

Said single-phase liquid phase 11 comprises water (notably the residual water from step b)), extraction solvent, and optionally the phthalates extracted in step a) and not converted, IAPs and ALs. Starting from said single-phase liquid effluent 11, it is then possible, according to separation methods well known to those skilled in the art, for example a distillation with a side stream or a liquid-liquid extraction, to separate, during step f1) preferably directly downstream of step e), not only the extraction solvent (stream 15) and the residual water (effluent 14), but also the ALs (stream 17) and very advantageously the IAPs with possibly the phthalates extracted in step a) and not converted (stream 16).

According to the first and second variants of the process according to the invention, stream 15 comprising the extraction solvent and stream 14 comprising water may then advantageously be sent back to step a) and to step b) of the process according to the invention respectively. Likewise, stream 16 comprising the IAPs with possibly the phthalates extracted in step a) and not converted is advantageously sent back to step b), so as to continue the chemical reactions resulting in PA and thus improve the yield of PA.

According to one variant (not shown), the extraction solvent may be extracted completely from stream 3 in step g), and step b) may be carried out by those skilled in the art so that the effluent leaving said step b) forms when the same liquid phase containing at least the residual water and PA, and potentially the phthalates extracted in step a) and not converted, the IAPs, and the ALs. The steps carried out downstream are identical to steps d), e) and f1) described above in connection with FIG. 1, except for the fact that the extraction solvent is no longer present downstream of step g), and therefore that there is no production of a stream 15 in step f1).

According to one or more embodiments of the invention, and provided that the extraction solvent has not been completely removed from stream 3 during step g) when it is carried out, said solvent and the operating conditions of the process of the invention are chosen so that said solvent and the water 9 form a first liquid phase of the effluent 4 containing at least PA, a second liquid phase that is immiscible with the first also being formed and comprising at least the phthalates extracted in step a) and not converted.

According to this or these embodiment(s), represented in FIGS. 2 and 5, it is possible to separate in a step f3), preferably directly upstream of step d), at least the two preceding immiscible phases so as to obtain at least one stream 18 comprising the first liquid phase containing the water and solvent and also the PA, and a stream 16 comprising the second liquid phase that is immiscible with the fast, which is an organic phase, containing at least the IAPs and the phthalates extracted in step a) and not converted.

The choice of a water-miscible solvent, such as MEK or a MEK/methanol mixture, is compatible with obtaining two immiscible liquid phases in effluent 4.

The stream 16 is advantageously sent back to step b) of the process according to the invention, so as to continue the chemical reactions resulting in PA and thus improve the yield of PA. The streams obtained on conclusion of step e) then comprise the solid stream comprising PA 6 and a liquid effluent 20 comprising residual water (from step b)), said liquid effluent 20 being in the form of a single-phase liquid phase comprising water and extraction solvent. Starting from said single-phase liquid effluent 20, it is then possible, according to separation methods well known to those skilled in the art, for example a distillation, to separate in a step f2), preferably directly downstream of step e), the extraction solvent (stream 15) and water (stream 14), which may advantageously be reused in step a) and in step b) respectively as described above in connection with FIGS. 1 and 4. According to this or these embodiment(s), and depending on their chemical nature, the Als may either be present in stream 16 resulting from the separation step f3) and/or stream 20 resulting from step e), and consequently be removed in the form of a stream 17 from the process, before recycling the various streams 14, 15 and 16.

According to one variant (not shown), the extraction solvent may be extracted completely from stream 3 in step g), and step b) may be carried out by those skilled in the art so that the effluent leaving said step b) comprises at least a first liquid phase containing at least PA and a second liquid phase that is immiscible with the first containing the phthalates extracted in step a) and not converted and the ALs. The steps carried out downstream are identical to steps d), e), f2) and f3) described in connection with FIG. 2, except for the fact that the extraction solvent is no longer present downstream of step g), and therefore that there is no production of a stream 15 in step f2).

According to one or more embodiments of the invention, and provided that the extraction solvent has not been completely removed from stream 3 in step g) when it is carried out, said solvent and the operating conditions of the process of the invention are chosen so that said solvent and the water 9 form two immiscible liquid phases of the effluent 4. The first of these liquid phases is an aqueous phase containing at least PA; it comprises the residual water from step b) and PA, and is immiscible with the extraction solvent. The second of these liquid phases is an organic phase comprising the extraction solvent, containing at least the phthalates extracted in step a) and not converted, IAPs and ALs.

According to this or these embodiment(s), represented in FIGS. 3 and 6, it is possible to separate in a step f4), preferably directly downstream of step d), at least the two preceding immiscible phases so as to obtain at least one stream 21 comprising the aqueous liquid phase containing the PA and at least one stream 23 comprising the organic phase containing the extraction solvent.

The choice of a water-immiscible extraction solvent, such as toluene, may enable the production of said first aqueous liquid phase which is immiscible with the organic liquid phase comprising the extraction solvent forming the effluent 4.

The implementation of steps d) and e) starting from stream 21 leads to the production of at least the solid stream comprising the PA 6 and of at least the aqueous stream 14, preferably sent back completely or partly to step b).

Starting from the organic liquid stream 23, it is possible, according to separation methods well known to those skilled in the art, for example well-known liquid-liquid and/or liquid-gas separation methods such as distillation, decantation, evaporation, liquid-liquid extraction, etc., performed alone or in combination, to separate, during step f5), not only the extraction solvent (stream 15), but also the ALs (stream 17) and very advantageously the IAPs with possibly the phthalates extracted in step a) and not converted (stream 16). The stream 16 may advantageously be sent back to step b), so as to continue the chemical reactions resulting in PA and thus improve the yield of PA. Similarly, the stream 15 may advantageously be sent back to step a).

### Recycling Process

The present invention also relates to a process for recycling a PVC-based object containing at least one phthalate, said recycling process including:
the conditioning of the PVC-based object comprising at least milling or shredding of the PVC-based object to form a PVC feedstock in the form of particles;
the recovery of the PA and of a reusable target PVC plastic from said PVC feedstock in the form of particles according to the process according to the first aspect of the invention described in detail above.

The step of conditioning of the PVC-based object may include the various steps detailed above for the preconditioning of the PVC feedstock before it is introduced into step a).

It is advantageous, from a circular economy point of view, to use the phthalic acid obtained by the recovery process described in order to again obtain phtalates suitable for the formulation of flexible PVC plastics and/or to use the target PVC plastic produced by the recovery process according to the invention in order to manufacture a new flexible PVC-based object. Such an object can then be manufactured more easily so as to meet the standards in force regarding phthalates, and to include only REACH-compatible phthalates, by being manufactured from raw materials respecting or adapted to respect said standards, i.e. the target PVC plastic recovered which is free of non-REACH-compatible phthalates, and the PA enabling the production of REACH-compatible phthalates.

### Manufacturing Process

The present invention also relates to a process for manufacturing a flexible PVC-based object comprising a recycled PVC plastic and/or a phthalate manufactured from phthalic acid recovered by the process according to the first aspect of the invention.

Such a manufacturing process typically comprises a step of recovering phthalic acid and a reusable target PVC plastic from a PVC feedstock, as detailed above, followed by a step of mixing said reusable target PVC plastic with additives, and then a step of forming said mixture.

### EXAMPLE

This example illustrates the invention without limiting the scope thereof, and notably illustrates the extraction of a phthalate contained in a PVC plastic and the conversion of the phthalate to phthalic acid in the presence of a catalyst and water.

18.2 g of a PVC plastic feedstock (obtained from PVC-based objects of “medical tubing” type), in the form of extrudates with a mean size of 2 mm, containing 4.4 g of didecyl phthalate (DIDP), are introduced into a reactor stirred with a mechanical stirring system, of paddle type. 53.19 g of water and 35.11 g of acetophenone (organic extraction solvent) then added, the acetophenone/water weight ratio being 0.66, the acetophenone/DIDP weight ratio being 29.7 and the water/DIDP mole ratio being 300. 0.13 g of catalyst, which is p-toluenesulfonic acid (APTS), are then added to the preceding mixture so that the APTS/DIDP weight ratio is 3%.

The reactor is hermetically closed, purged with nitrogen and then heated to 100° C. with an autogenous pressure of the order of 1.2 MPa and maintained under these conditions for 4 hours with stirring of 1000 rpm.

After 4 hours, a solid and a liquid are obtained which are separated while hot at 65° C., which makes it possible to obtain a PVC solid with a very low content of phtalates on the one hand and a liquid on the other hand. This liquid is then cooled to 10° C., which leads to the precipitation of a second solid predominantly consisting of phthalic acid. The suspension obtained is filtered. The remaining liquid (filtrate) is then again left three times under the same conditions as described previously. At the end of each reactive step, a precipitation step, identical to that specified above, makes it possible to extract a solid phase predominantly consisting of phthalic acid, increasing the final phthalic acid yield. At the end of this protocol, the secondary solid fractions are collected together and analysed.

The analyses by gas chromatography with flame ionization detection (GC-FID) of the secondary solid phase show that it contains 1.47 g of phthalic acid resulting from the conversion of the DIDP and 0.30 g of monomethyl phthalate (2-(isodecoxycarbonyl)benzoic acid) resulting from a partial hydrolysis of the DIDP. The liquid for its part contains 2.96 g of isodecanol (C₁₀H₂₂₀) resulting from the DIDP hydrolysis reaction. Identification was made possible by comparison of the retention times of pure analytical standards and quantification was performed by determination of the response coefficients derived from the analysis of these same standards.

The solid obtained was prefractionated by preparative size exclusion chromatography SEC equipped with double optical detection (UV/visible) and refractometry (RI). The fractions obtained from the collection were analyzed by high-performance liquid chromatography (HPLC) equipped with optical detection of quantitative UV-visible type The results indicate the presence of DIDP in the target PVC plastic in a content of less than 1000 ppm, which complies with the European regulations in force.

These results show that a phthalate-free PVC in accordance with the invention is obtained, and that the DIDP was 99.9% converted. In this example, the extraction of the DIDP and its conversion are performed in the same step.

1. Process for recovering phthalic acid and a reusable target PVC plastic from a PVC feedstock containing at least one phthalate, including the following steps:
a) a solid-liquid extraction of said PVC feedstock in the form of particles (1) by placing said particles of the PVC feedstock in contact with at least one organic solvent (7) for extracting said phthalate, to produce a liquid phase enriched in said phthalate and a first solid phase comprising PVC plastic depleted in said phthalate;
b) a chemical conversion of said phthalate extracted in step a) to phthalic acid of formula C₆H₄ (COOH)₂ by hydrolysis using water to produce an aqueous phase comprising said phthalic acid;
c) a solid-liquid extraction between said first solid phase and said liquid phase enriched in said phthalate or said aqueous phase comprising said phthalic acid to produce at least one solid stream comprising the PVC plastic depleted in said phthalate (8) in order to recover said target PVC plastic;
d) a phase change of the phthalic acid from the dissolved state in said aqueous phase to a solid state to produce a mixed stream comprising an aqueous liquid phase depleted in phthalic acid and a second solid phase enriched in phthalic acid (5, 19, 22);
e) a solid-liquid separation between the phthalic acid in the solid state from step d) and the aqueous liquid phase of said mixed stream to produce a solid stream of phthalic acid (6) and a liquid effluent (11, 14, 20) comprising residual water from step b).

2. Process according to claim 1, in which steps a) and b) are carried out within the same individual operation producing a stream (24) comprising at least said aqueous phase comprising said phthalic acid and said first solid phase.

3. Process according to claim 1, in which steps a) and b) form the subject of two distinct individual operations, step a) producing a stream (2) comprising said liquid phase enriched in said phthalate and said first solid phase sent to step c) carried out between steps a) and b), step c) producing said stream comprising the PVC plastic depleted in said phthalate (8) and a first liquid stream (3) comprising said liquid phase enriched in said phthalate.

4. Process according to claim 3, comprising a step g) for removing at least one portion of the organic extraction solvent from said first liquid stream (3) prior to step b), to produce a first concentrated liquid stream comprising said phthalate sent to step b).

5. Process according to claim 4, in which the organic extraction solvent recovered in step g) is recycled at least in part to step a).

6. Process according to any claim 1, in which said solid stream comprising the phthalate-depleted PVC plastic (8) separated in step c) is recycled at least in part to step a).

7. Process according to claim 1, in which said organic extraction solvent (7) is chosen from the list consisting of ketones, ethers, glycol ethers, cyclic and aromatic hydrocarbons, alcohols with a linear or branched short aliphatic chain, of empirical formula CₙH₂ₙ₊₁OH with n<4, n being a non-zero natural integer, and mixtures thereof.

8. Process according to claim 7, in which said organic extraction solvent (7) is chosen from the list consisting of methyl ethyl ketone, diethyl ketone, 4-heptanone, 2,4-dimethyl-3-pentanone, methyl isobutyl ketone, cyclopentanone, cyclohexanone, acetophenone, tetrahydrofuran, cyclohexane, xylenes, toluene, methanol, ethanol, n-propanol, i-propanol, and mixtures thereof, and preferably is chosen from said alcohols, ketone/alcohol mixtures, hydrocarbon/alcohol mixtures, cyclohexane, toluene, xylenes, methoxycyclopentane, methyl isobutyl ketone, cyclopentanone, acetophenone, and preferably said organic extraction solvent (7) is toluene, methyl ethyl ketone, acetophenone or a methyl ethyl ketone/methanol mixture.

9. Process according to claim 1, in which the hydrolysis in step b) is carried out in the presence of an acid hydrolysis catalyst, preferably a homogeneous acid catalyst chosen from the list consisting of mineral Brönsted acid catalysts, preferably hydrochloric acid, sulfuric acid or phosphoric acid, organic Brönsted acid catalysts, preferably p-toluenesulfonic acid, and Lewis acid catalysts, preferably AlF₃, or a heterogeneous acid catalyst chosen from the list consisting of aluminas, chlorinated aluminas, fluorinated aluminas, mesoporous aluminosilicates, zeolites and mixtures thereof with other oxides, (H+) ion-exchange resins, preferably sulfonic resins.

10. Process according to claim 1, in which the hydrolysis in step b) is carried out at a temperature of between room temperature and 150° C., preferably between 40° C. and 130° C., at a pressure of between atmospheric pressure and 5.0 MPa, preferably between atmospheric pressure and 2.0 MPa, and for a time of between 1 minute and 10 hours, preferably between 10 minutes and 4 hours.

11. Process according to claim 1, in which the hydrolysis in step b) is carried out so that the mole ratio of the amount of water to the amount of said phthalate extracted in step a) is between 100 and 9000.

12. Process according to claim 1, in which step d) comprises at least one step of precipitating the phthalic acid, preferably comprising a cooling to a temperature between 10° C. and room temperature.

13. Process according to claim 1, in which said at least one phthalate of said PVC feedstock is a phthalate of empirical formula C₆H₄ (COOR₁) (COOR₂) in which the ester groups are in the ortho position of the benzene ring, R₁ or R₂ being chosen independently from one of the elements of the group consisting of a linear or branched or cyclic alkyl chain, a linear or branched alkoxyalkyl chain, or an aryl or alkylaryl chain, R₁ and/or R₂ preferably comprising between 1 and 20 carbon atoms, or even between 1 and 15 carbon atoms.

14. Process according to claim 1, in which said target PVC plastic is free of said phthalate, and preferably comprises less than 0.1% by weight in total of phthalates chosen from the list consisting of dibutyl phthalate, dioctyl phthalate or diethylhexyl phthalate, benzyl butyl phthalate, dibutyl phthalate, diisobutyl phthalate, dipentyl phthalate, diisopentyl phthalate, n-pentyl isopentyl phthalate, dihexyl phthalate, bis(2-methoxyethyl) phthalate, and mixtures thereof.

15. Process for recycling a PVC-based object containing at least one phthalate, including:
the conditioning of said PVC-based object comprising at least milling or shredding of said PVC-based object to form a PVC feedstock in the form of particles;
the recovery of phthalic acid and of a reusable target PVC plastic from said PVC feedstock in the form of particles according to claim 1.
